(19) United States
(12) Patent Application Publication
(10) Pub. No.: US 2026/0035322 A1
(43) Pub. Date: Feb. 5, 2026

(54) PROCESS FOR INCREASING THE HYDROGEN PURITY AND RECOVERY IN DEHYDROGENATION

(71) Applicant: UOP LLC, Rosemont, IL (US)

(72) Inventors: Srinivasan Ramanujam, Bengaluru (IN); Niki Naik Shah, Gurugram (IN); Ian G. Horn, Streamwood, IL (US); Sudipta K. Ghosh, Gurgaon (IN)

(21) Appl. No.: 19/246,432

(22) Filed: Jun. 23, 2025

Related U.S. Application Data
(60) Provisional application No. 63/678,036, filed on Jul. 31, 2024.

(51) Int. Cl. C07C 5/393; C07C 7/09

(52) U.S. Cl. CPC C07C 5/393; C07C 7/09

(57) ABSTRACT

Present disclosure relates to process for increasing the purity and recovery of hydrogen product stream obtained from a dehydrogenation process. The dehydrogenated effluent stream is separated to obtain the net gas stream. The net gas stream is compressed in a hydrogen compressor section. Even after compressing the net gas, traces of toluene are still present in the gas phase. Applicants have found to recover these toluene traces, the compressed gas from the hydrogen compressor section is chilled in a vapor economizer followed by a chiller to produce a chilled stream at a temperature of from about −27° C. to about 17° C. This process also helps maintain the Wobbe Index of the hydrogen product stream in the range of about 44 to about 49 MJ/m³. In another embodiment, the compressed net gas stream from the hydrogen compressor section is adsorbed in an adsorption unit to separate hydrogen product stream from the recycle gas stream, wherein the recycle gas stream is completely recycled to the hydrogen compressor.

### FIELD

The subject matter of the present disclosure generally relates to processes and apparatuses for increasing the purity and recovery of hydrogen product stream in a dehydrogenation process.

### BACKGROUND

Hydrogen is expected to have significant growth potential because it is a clean burning fuel. Aggressive carbon emission reduction targets and rising carbon penalties to meet the Paris Climate Agreement are anticipated to drive a hydrogen-based economy in the near future.

However, hydrogen production processes based on steam reforming, autothermal reforming, partial oxidation, or gasification of hydrocarbon or carbonaceous feedstocks are significant emitters of carbon dioxide. Government regulations and societal pressures are increasingly taxing or penalizing carbon dioxide emissions or incentivizing carbon dioxide capture. Consequently, there is significant interest in lowering the cost of hydrogen production using these processes and recovering the byproduct carbon dioxide for subsequent geological sequestration. Hydrogen from solar, wind, and water known as green hydrogen, which does not involve the production of carbon dioxide and could meet projected global energy demand in the future and play a vital role in reducing carbon dioxide emissions. The recently renewed interest in alternative energy sources and energy carriers open up new prospects, for example in fuel cells, power generation and many more applications.

There exists a huge regional disparity in the cost of production of hydrogen. Several technologies have been developed for transporting hydrogen, including ammonia, liquid hydrogen, and liquid organic hydrogen carrier (LOHC) to address this disparity. Toluene to methylcyclohexane (MCH) is expected to be a significant player in the LOHC space considering numerous advantages, such as easy integration with existing fuel sector supply chain and distribution network, utilization in idle refinery assets, flexibility for co-processing, and higher relative handling safety.

The process using LOHC involves hydrogenation and dehydrogenation cycle to transport hydrogen. Toluene to methylcyclohexane (MCH) LOHC involves hydrogenation of toluene to MCH at the hydrogen source and dehydrogenation of MCH back to hydrogen and toluene at its destination.

The hydrogen product produced after dehydrogenation may have a number of different end destinations with different purity requirements. The commonly used destinations are fuel cell and power generation for buildings. The purity requirement for fuel cells and power generation units are very stringent. Due to the relatively high cost of hydrogen produced by LOHC, it is necessary to recover almost all hydrogen.

In addition to high purity hydrogen, additional requirements for fuel cell applications are recovering trace amounts of toluene that are lost with the hydrogen product stream, and also maintaining the Wobbe Index which is an indicator of the interchangeability of fuel gases. The Wobbe Index, IW, is the calorific value of the gas divided by the square root of its specific gravity. Recovering toluene saves the make-up cost of initial reactants in the hydrogenation step. In addition to high purity, an additional requirement for a building power generation application is reducing the aromatics in the hydrogen product stream to less than about 1 wt to about 25 wt ppm.

Accordingly, it would be desirable to produce a high-purity hydrogen product stream to meet the requirements in different applications and avoid unnecessary operating or capital costs in the purification process.

### SUMMARY

A process for increasing the purity and recovery of hydrogen product stream in the dehydrogenation process is provided. A hydrogenated hydrocarbon such as MCH is transferred in storage vessels, tankers and/or pipelines for several thousands of miles to a final destination with very minimal to no degradation. At the destination the hydrogenated hydrocarbon is dehydrogenated to produce a dehydrogenated hydrocarbon such as toluene and a hydrogen product stream. The objective of the present disclosure is to increase the purity of hydrogen product stream, remove the undesirable aromatics from the hydrogen product stream and also meet a Wobbe Index for the hydrogen product stream in a range of about 44 to about 49 MJ/m3.

In first aspect of the invention, in addition to higher hydrogen purity, the objective is to recover about 1 wt % toluene that is lost in the gaseous phase of hydrogen product stream and also maintain the Wobbe Index in the desired range. Recovering toluene helps improve the purity of hydrogen product stream and saves cost of the makeup toluene purchase required for the hydrogenation unit. Applicants have found that by chilling the compressed gas from the net gas section to about −27° C. to about 17° C., preferably about −27° C. to about 14° C., and more preferably about −27° C. to about 10° C., more preferably about −27° C. to about 4° C., helps to recover the 1 wt % toluene and while maintaining the desired range of the Wobbe Index of the hydrogen product stream. The chilling may take place in an economizer and chiller system.

Another aspect of the invention is to remove aromatics from the hydrogen product stream. The objective is to increase hydrogen purity to at least 99.7 mol % and limit aromatics to less than 1 wtppm of BTX (benzene-toluene-xylene). One way to meet this specification is using an adsorption step. The adsorption step can be carried out in a pressure swing adsorption (PSA) unit or a temperature swing adsorption (TSA) unit. The hydrogen recovery in the adsorption unit is about 90%, so to increase the hydrogen recovery, 100% of the recycle gas from the adsorption unit is recycled to a hydrogen compressor section inlet. Applicants have also found, when the separator operates at a pressure of less than 60 psig (414 kPa), preferably less than 50 psig (345 kPa), then tail gas from the PSA unit is completely recycled without the need for a tail gas compressor, eliminating the capital cost of this unit.

### Definitions

As used herein, “liquid organic hydrogen carrier” or “LOHC” refers to a hydrogenated organic substrate selected from monocyclic, polycyclic, heterocyclic and homocyclic organic compounds that can be processed to release chemically bound hydrogen via dehydrogenation and which are liquid at standard temperature and pressure.

As used herein, the term “wt %” as used here is equivalent to “percent by weight”.

As used herein, the acronym “BTX” is intended to refer to a mixture of benzene, toluene, and xylene, in any ratio.

As used herein, “Wobbe Index” is the measure of fuel quality. The performance of different fuels can be compared using a parameter known as the Wobbe Index. The Wobbe Index (Iw) is defined by the following equation:

$${I}_{W} = \frac{{V}_{C}}{\sqrt{{G}_{S}}}$$

where the gross heating value (Vc) is also known as the higher heating value of the fuel and Gs is gas specific gravity. It is measured in MJ/m³.

As used herein, the acronym “PSA” is intended to refer to Pressure Swing Adsorption unit. PSA refers to a process where a contaminant is adsorbed from a gas when the process is under a relatively higher pressure and then the contaminant is removed or desorbed thus regenerating the adsorbent at a lower pressure.

As used herein, the acronym “TSA” is intended to refer to Temperature Swing Adsorption unit. TSA refers to a process where regeneration of the adsorbent is achieved by an increase in temperature such as by sending a heated gas through the adsorbent bed to remove or desorb the contaminant. Then the adsorbent bed is often cooled before resumption of the adsorption of the contaminant.

As used herein, the term “separator” means a vessel which has an inlet and at least an overhead vapor outlet and a bottoms liquid outlet and may also have an aqueous stream outlet from a boot.

As used herein, the term “portion” means an amount or part taken or separated from a main stream without any change in the composition as compared to the main stream. Further, it also includes splitting the taken or separated portion into multiple portions where each portion retains the same composition as compared to the main stream.

As used herein, the term “zone” or “unit” or “section” can refer to an area including one or more equipment items and/or one or more sub-zones. Equipment items can include one or more reactors or reactor vessels, heaters, discharge drums, exchangers, pipes, pumps, compressors, and controllers. Additionally, an equipment item, such as a reactor, dryer, or vessel, can further include one or more zones or sub-zones.

As used herein, the term “overhead stream” can refer to a stream withdrawn at or near a top of a vessel, such as a column.

As used herein, the term “bottoms stream” can refer to a stream withdrawn at or near a bottom of a vessel, such as a column.

As used herein, the term “economizer” can refer to a heat exchanger that exchanges heat between two streams.

As used herein, the term “chiller” can refer to a machine, comprising a condenser and evaporator, that removes heat from a stream using a refrigerant.

As used herein, the term “communication” means that fluid flow is operatively permitted between enumerated components, which may be characterized as “fluid communication”.

As used herein, the term “downstream communication” means that at least a portion of fluid flowing to the subject in downstream communication may operatively flow from the object with which it fluidly communicates.

As used herein, the term “direct communication” or “directly” means that fluid flow from the upstream component enters the downstream component without passing through any other intervening vessel.

### BRIEF DESCRIPTION OF THE DRAWINGS

FIG. 1 is an illustration of one embodiment of a process of dehydrogenating MCH comprising a chiller and an economizer in the net gas section.

FIG. 2 is an illustration of one embodiment of a process of dehydrogenating MCH comprising an adsorption unit in the net gas section.

### DETAILED DESCRIPTION

Applicants have discovered processes and apparatuses for increasing the purity and recovery of a hydrogen product stream recovered from dehydrogenation unit. Applicants have found by chilling a compressed gas stream from a hydrogen gas compressor in a vapor economizer and chiller helps recover the dehydrogenated hydrocarbon and maintain Wobbe index in the desired range. In another embodiment compressed gas from the net gas compressor is purified using an adsorption unit. The process comprises dehydrogenating a hydrogenated stream received from the storage or transport to produce a dehydrogenated effluent stream. The dehydrogenated effluent stream is separated to obtain the net gas stream. The net gas stream is compressed in a hydrogen compressor section. Even after compressing the net gas, traces of toluene are still present in gaseous hydrogen stream. Applicants have found that to recover these toluene traces, the compressed gas from the hydrogen compressor section may be chilled in a vapor economizer followed by a chiller to produce a chilled stream at a temperature of from about −27° C. to about 17° C., preferably about −27° C. to about 10° C., more preferably about −27° C. to about 4° C. This process also helps maintain the Wobbe Index of the hydrogen product stream in the range of about 44 to about 49 MJ/m3.

In another aspect, the compressed net gas stream from the hydrogen compressor section is adsorbed in an adsorption unit to separate a hydrogen product stream from a recycle stream. In an embodiment, the adsorption unit can be a PSA unit. In another embodiment, the adsorption unit can be a TSA unit. The recycle stream is completely recycled to the hydrogen compressor. Alternatively, the recycle stream can also be recycled to the separator overhead stream. The PSA unit operates at a pressure of about 35 bar (g) (510 psig) to about 80 bar (g) (1160 psig), preferably about 35 bar (g) (510 psig) to 50 bar (g) (730 psig), at the feed inlet. And in case the separator operates at a pressure of less than 50 psig (345 kPa (g)), tail gas stream can be recycled without the need of a tail gas compressor. The TSA unit operates at a regeneration temperature of about 200° C. (390° F.) to about 450° C. (840° F.), preferably about 200° C. (390° F.) to 300° C. (570° F.).

As depicted, process flow lines in the FIGURE can be referred to, interchangeably, as, e.g., lines, pipes, branches, distributors, streams, effluents, feeds, products, portions, catalysts, withdrawals, recycles, suctions, discharges, and caustics.

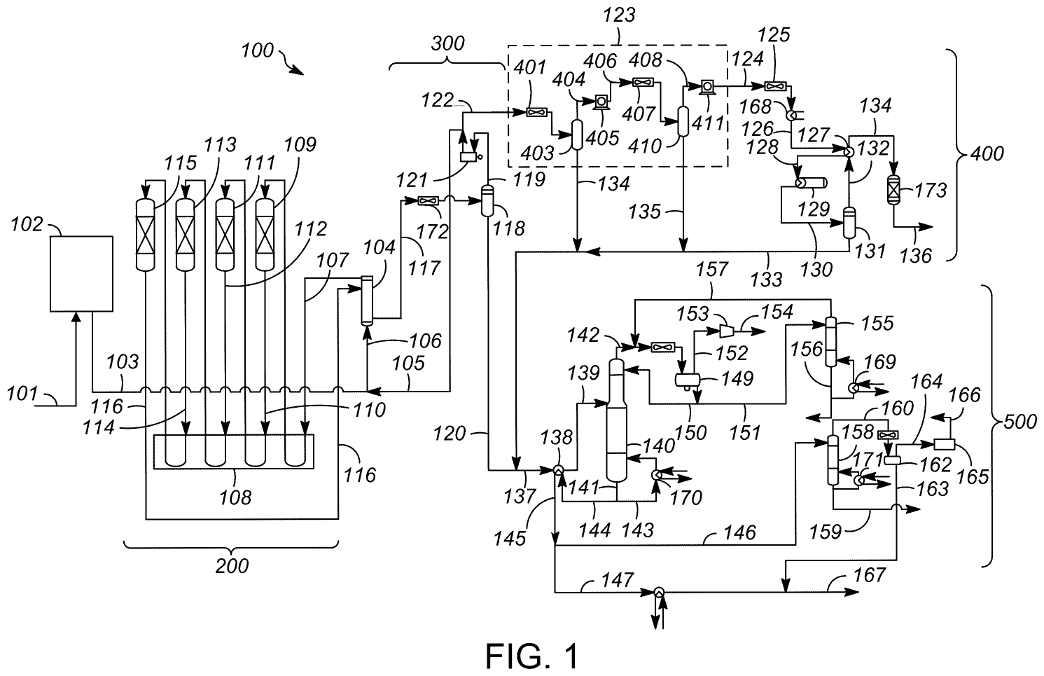

FIG. 1 illustrates an embodiment of the process 100 of purifying hydrogen product stream, comprising a chiller and an economizer in a net gas section. The process 100 comprises a dehydrogenation reactor zone 200, a separation section 300, a net gas section 400 and a toluene recovery section 500. As shown, a hydrogenated stream perhaps comprising methylcyclohexane stream in line 101 is taken from an external source such as a storage tank or a transport tanker (not shown). If the hydrogenated feed stream is imported from an external source, such as through a pipeline, land-going vehicle, or water-going vehicle, the feed stream may be exposed to oxygen and require treatment to remove the oxygen and/or oxygenated hydrocarbons prior to introduction into the dehydrogenation reactor zone 200. In an exemplary embodiment, the hydrogenated feed stream in line 101 may be passed to an oxygen stripper 102 to remove one or both of oxygen and oxygenated hydrocarbons and produce a deoxygenated stream in line 103. The hydrogenated feed stream in line 101 may comprise methylcyclohexane. In an aspect, hydrogenated feed stream in line 101 may comprise a product of a hydrogenation unit. The oxygen stripper 102 may remove any water present in the hydrogenated feed stream in line 101.

The deoxygenated stream in line 103 is combined with a recycle hydrogen stream in a line 105 to provide a combined feed stream in line 106. In an embodiment, the hydrogenated feed stream in line 101 is combined with a recycle hydrogen stream in a line 105 to provide a combined feed stream in line 106. The combined feed stream in line 106 is preheated in a combined heat exchanger 104 to provide a preheated combined stream in line 107. In an aspect, the hydrogenated feed stream in line 101 and the recycle hydrogen stream in a line 105 may be preheated separately to provide a preheated hydrogenated feed stream and a preheated recycle hydrogen stream. The preheated hydrogenated feed stream and the preheated recycle hydrogen stream may be charged to one or more dehydrogenation reactors.

The dehydrogenation reaction zone 200 comprises a dehydrogenation reactor(s) and a process heater 108. In an exemplary embodiment, the dehydrogenation reaction zone 200 comprises four dehydrogenation reactors, a first dehydrogenation reactor 109, a second dehydrogenation reactor 111, a third dehydrogenation reactor 113, and a fourth dehydrogenation reactor 115. Fewer or more dehydrogenation reactors than four may be utilized.

The preheated combined stream in line 107 is heated in a first loop of a process heater 108 and charged to the first dehydrogenation reactor 109 to produce a first dehydrogenated effluent stream in line 110. The first dehydrogenated effluent stream in line 110 is heated in a second loop of the process heater 108 and is charged to the second dehydrogenation reactor 111 to produce a second dehydrogenated effluent stream in line 112. The second dehydrogenated effluent stream 112 is heated in a third loop of the process heater 108 and is charged to the third dehydrogenation reactor 113 to produce a third dehydrogenated effluent stream in line 114. The third dehydrogenated effluent stream in line 114 is heated in a fourth loop of the process heater 108 and is charged to the fourth dehydrogenation reactor 115 to produce a dehydrogenated effluent stream in line 116. Process heater 108 raise the effluent back to the desired reactor inlet temperature between dehydrogenation reactor(s).

The dehydrogenation reactor(s) are typically downflow or radial flow reactors. Suitable dehydrogenation catalysts may include, but are not limited to, alumina, a noble metal, and an alkali or alkaline earth metal. Suitable noble metals include, but are not limited to, platinum, palladium, rhodium, ruthenium, rhenium, iridium, gold, osmium, silver, or combinations thereof. Suitable alkali or alkaline earth metals include, but are not limited to, sodium, cesium, potassium, rubidium, francium, lithium, beryllium, strontium, barium, calcium, magnesium, radium, or combinations thereof. The dehydrogenation reactor operates at a temperature of about 420° C. (788° F.) to about 600° C. (1112° F.), preferably about 480° C. (896° F.) to about 600° C. (1112° F.), more preferably about 500° C. (932° F.) to about 600° C. (1112° F.) and pressure of about 90 psig (620 kPa (g)) to 110 psig (758 kPa (g)).

The dehydrogenated effluent stream in line 116 exchanges heat with the combined feed stream in line 106, in the combined heat exchanger 104. The dehydrogenated effluent stream in line 116 from the fourth dehydrogenation reactor 115 of the dehydrogenation reaction zone 200 is cooled in the combined heat exchanger 104 to provide a cooled dehydrogenated effluent stream in line 117.

Generally, the combined heat exchanger heats the combined feed stream by transferring heat from effluent stream of the last reactor of the dehydrogenation reaction zone to the combined feed stream. Preferably, the combined heat exchanger is an indirect, rather than a direct, heat exchanger, in order to prevent intermixing of the dehydrogenated effluent stream with the combined stream. Although shell-and-tube type heat exchangers may be used, another possibility is a plate type heat exchanger. Plate type exchangers are well known and commercially available in several different and distinct forms, such as spiral, plate and frame, brazed-plate fin, and plate fin-and-tube types. Plate type exchangers are described generally on pages 11-52 to 11-53 in Perry's Chemical Engineers' Handbook, Seventh Edition, edited by R. H. Perry et al., and published by McGraw Hill Book Company, in New York, in 1984.

The cooled dehydrogenated effluent stream in line 117 is separated in the separation section 300. In an embodiment, the cooled dehydrogenated stream in line 117 is further cooled in a product condenser 172 prior to the separation section 300. The separation section comprises a separator 118 and a recycle compressor 121. The cooled dehydrogenated effluent stream in line 117 is sent to the separator 118 to provide a separator overhead stream in line 119 comprising hydrogen and a separator bottom stream in line 120 comprising dehydrogenated hydrocarbon. The separator 118 is a gas-liquid separator that operates at a pressure of about 70 psig (483 kPag) to about 110 psig (758 kPag), preferably to about 70 psig (483 kPag) to about 90 psig (621 kPag) and a temperature of about 20° C. (68° F.) to about 60° C. (140° F.), preferably about 40° C. (104° F.) to about 50° C. (122° F.). In an embodiment the separator operates at a pressure of less than 60 psig (414 kPag), preferably less than 50 psig (345 kPag).

The separator overhead stream in line 119 is compressed in the recycle compressor 121 to obtain the recycle hydrogen stream in line 105 and a net gas stream in a net gas line 122. The recycle hydrogen stream in line 105 is recycled to the combined heat exchanger 104 to be mixed with the deoxygenated stream in line 103. The recycle hydrogen stream in line 105 helps maintain partial pressure of hydrogen in the dehydrogenation reaction zone 200.

The net gas stream in the net gas line 122 is sent to a hydrogen compressor section 123 to provide a compressed stream in a compressed line 124, a first liquid stream in line 134 and a second liquid product stream in line 135.

The hydrogen compressor section 123, as described in detail below, comprises one or more compressor(s) which provide sufficient pressure to meet the hydrogen purity requirements of the user. However, compression is not sufficient to meet the stringent purity requirements and remove the traces of toluene from the gaseous phase. The hydrogen compressor section 123 receives the net gas stream in the net gas line 122 from the recycle compressor 121. The net gas stream in line 122 is cooled in a trim cooler 401 and then is separated in a suction drum 403 to produce a suction drum overhead stream in line 404 and the first liquid product in line 134. The suction drum overhead stream in line 404 is compressed in a first stage compressor 405 to produce a first stage compressed stream in line 406. The first stage compressed stream in 406 is cooled in cooler 407 before being separated in a first stage discharge drum 410. The first discharge drum 410 produces a first discharge drum overhead stream in line 408 and the second liquid product stream in line 135. The first stage discharge drum overhead stream in line 408 is compressed in a second stage compressor 411 to produce the compressed stream in line 124. The stages of compressors can be increased or decreased based on the requirement of the plant. The first stage compressor operates at a discharge pressure of about 200 psig (1379 kpag) to about 300 psig (2068 kpag), preferably about 230 psig (1586 kpag) to about 300 psig (2068 kpag). The second stage compressor operates at a discharge pressure of about 400 psig (2758 kpag) to about 1000 psig (6895 kpag), preferably about 400 psig (2758 kpag) to about 600 psig (4137 kpag).

The compressed stream in the compressed line 124, is sent to a vapor economizer 127 to provide a cooled compressed stream in line 128. In an embodiment, the compressed stream in compressed line 124 is sent to a discharge cooler 125 and a trim cooler 168 prior to the vapor economizer 127 to condense some vapors in the compressed stream to provide two phase compressed stream in line 126. The two-phase compressed stream comprises liquid as well as vapor. In the vapor economizer 127, the two-phase compressed stream in line 126 is cooled by heat exchange with a discharge drum overhead stream in line 132 to produce a cooled compressed stream 128. The discharge drum overhead stream in line 132 is heated, while the two-phase compressed stream in line 126 is cooled. The cooled compressed stream 128 is further chilled in a chiller 129 to produce a chilled stream in line 130 at a temperature of about −27° C. to about 17° C., preferably about −27° C. to about 14° C., more preferably about −27° C. to about 10° C., more preferably about −27° C. to about 4° C. Applicants have found chilling the two phase compressed stream helps increase the purity of hydrogen and recover the dehydrogenated hydrocarbon which would otherwise be lost with hydrogen in the gaseous phase.

The chiller 129 operates using a refrigerant package, for example, comprising a refrigerant evaporator, compressor, condenser, and expansion valve or if desired, a more complex cascade system may be employed.

The chilled stream in line 130 is separated in a discharge drum 131 to produce the discharge drum overhead stream in line 132 and a discharge drum bottom stream in line 133. The discharge drum overhead stream in line 132 comprising hydrogen is passed to the vapor economizer 127. In the vapor economizer 127, heat exchange between the two-phase compressed stream in line 126 and the discharge drum overhead stream in line 132 takes place to produce a vapor economizer outlet stream in line 134. The two-phase compressed stream in 126 is cooled, while the discharge drum overhead stream in line 132 is heated. This heat exchange helps reduce energy consumption of refrigerant. The vapor economizer outlet stream in line 134 is further treated in a chloride treater 173 to obtain high purity hydrogen product stream in line 136. In an aspect, the vapor economizer outlet stream in line 134 may be treated in a sulfur guard bed to meet the hydrogen sulfide specification of the hydrogen product stream in line 136.

The separator bottom stream in line 120, a first liquid stream in line 134, a second liquid product stream in line 135, and the discharge drum bottom stream in line 133 are combined to form a combined liquid stream in line 137. The first liquid product in line 134 and the second liquid product stream in line 135 are obtained from the hydrogen compressor section 123. The combined liquid stream in line 137 is sent to a deheptanizer column 140 to recover toluene. In an embodiment, the combined liquid stream in line 137 is heated in a heat exchanger 138 before being passed to the deheptanizer column 140. A heated combined liquid stream may be taken in line 139 from the heat exchanger 138 and fed to the deheptanizer column 140. The deheptanizer column 140 produces a deheptanizer overhead stream in line 142 and a deheptanizer bottom stream in line 141. In an aspect, the separator bottom stream in line 120 may be fed to a hydrogenation unit to provide the hydrogenated feed stream in line 101.

The deheptanizer bottom stream in line 141 is split into two streams, a first stream in line 144 and a second stream in line 143. The first stream in line 144 exchanges heat with the combined liquid stream in line 137 in the heat exchanger 138 to produce cooled deheptanizer bottom stream in line 145. The second stream in line 143 is returned to the deheptanizer column 140 via a reboiler 170. The cooled deheptanizer bottom stream in line 145 is split into two streams, a first stream in line 146 and a second stream in line 147.

The deheptanizer overhead stream in line 142 is condensed and passed to a receiver 149 to produce a vapor stream in line 152, a reflux stream in line 150 and a distillate stream in line 151. The vapor stream in line 152 is passed to vent gas compressor 153 to produce an off gas stream in line 154. The off gas stream can be sent to a fuel gas header (not shown). The reflux stream in line 150 is returned to the top of the deheptanizer column 140. The distillate stream in line 151 is further stabilized in a stabilizer 155 to produce a stabilizer overhead stream 157 and a stabilizer bottom stream in line 156. The stabilizer overhead stream 157 is separated in the reflux drum 149 along with the deheptanizer overhead stream in line 142. The stabilizer bottom stream in line 156 comprises gasoline blending product. A portion of the stabilizer bottom stream in line 156 is vaporized in a reboiler 169 and returned to the bottom of the stabilizer 155.

The first stream in line 146 taken from the deheptanizer bottom stream in line 145 is further sent to a rerun column 158. The rerun column 158 produces a rerun overhead stream in line 160 and a rerum bottom stream in line 159. The rerun column bottom stream in line 159 comprises diesel blending product. A portion of the rerun column bottom stream 159 is vaporized in a reboiler 171 and returned to the bottom of the rerun column 158. The rerun overhead stream in line 160 is condensed and separated in a receiver 162 to produce a vapor stream in line 164 and a liquid stream in line 163. A vacuum pump 165 may be provided at the overhead of the rerun column 158 on the vapor stream in line 164 from the receiver 162 to operate the rerun column 158 at vacuum pressure. The vacuum pump 165 provides a rerun column off gas stream in line 166, which may be sent to a thermal oxidizer for destruction. The liquid stream in line 163 from the receiver 162 and the second stream of the cooled deheptanizer bottom stream in line 147 together provide the total recovered toluene product in line 167.

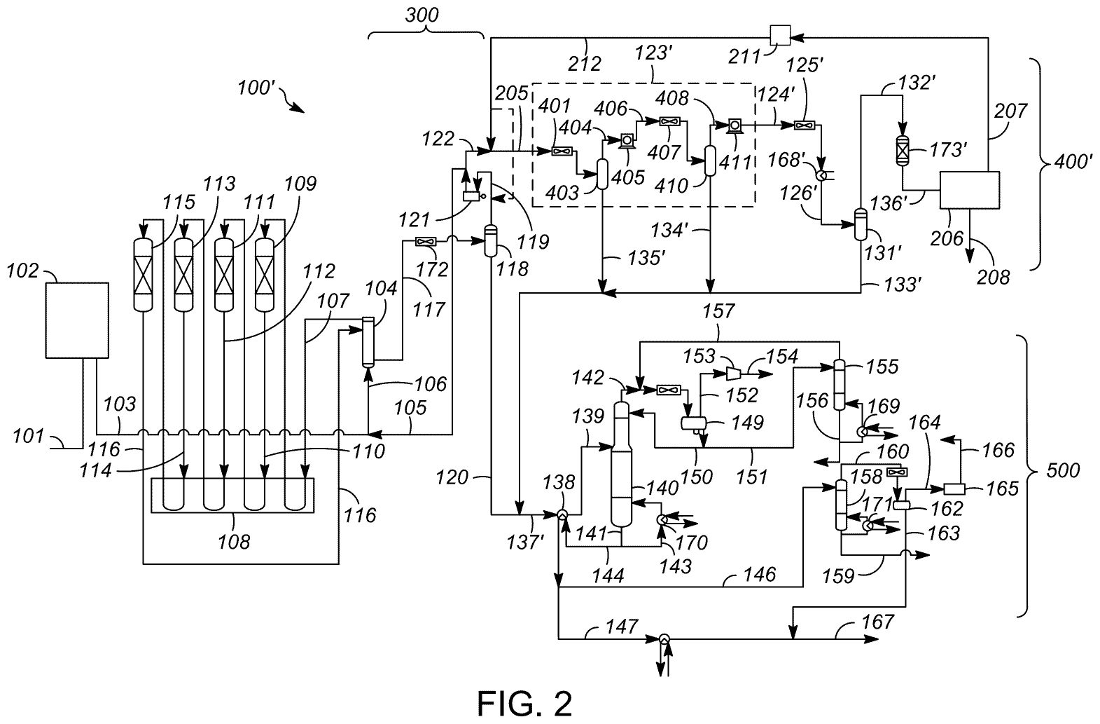

FIG. 2 shows an alternate embodiment of the process and apparatus of FIG. 1 in which the net gas section 400 is modified. The chiller 129 and the economizer 127 in the net gas section are removed, and an adsorption unit 206 is employed instead. Elements in FIG. 2 with the same configuration as in FIG. 1 will have the same reference numeral as in FIG. 1. Elements in FIG. 2 which have a different configuration as the corresponding element in FIG. 1 will have the same reference numeral but designated with a prime symbol (′). The configuration and operation of the embodiment of FIG. 2 is similar to FIG. 1 with the following exceptions.

In FIG. 2, the net gas stream in net gas line 122 along with a recycle stream in line 207 in a combined recycle stream in line 205 is compressed in a hydrogen compressor section 123′ to provide a compressed stream in a compressed line 124′.

The hydrogen compressor section 123′, as described in detail above, comprises one or more compressor(s) which provide sufficient pressure to meet the hydrogen purity requirements of the user. However, compression is not sufficient to meet the stringent purity requirements and remove the traces of toluene from the gaseous phase.

In an embodiment, the compressed stream in line 124′ is sent to a discharge cooler 125′ and a trim cooler 168′ to provide two phase compressed stream in line 126′. The two-phase compressed stream in line 126′ is separated in a discharge drum 131′ to provide a discharge drum overhead stream in line 132′ and a discharge drum bottom stream in line 133′.

The discharge drum overhead stream in line 132′ is treated in a chloride treater 173′ to remove the chlorides. The chloride treater 173′ produces a treated stream in line 136′.

The treated stream in line 136′ is sent to an adsorption unit 206 containing an adsorbent selective for the separation of hydrogen from BTX aromatics. In an embodiment, the adsorption unit can be a PSA unit. In another embodiment, the adsorption unit can be a TSA unit. The adsorption unit produces a hydrogen product stream in line 208 and a recycle stream in line 207. The recycle stream in line 207 is recycled to the hydrogen compressor section 123′. In an embodiment, the recycle stream in line 207 is recycled to the net gas stream in line 122. In another embodiment, the recycle stream in line 207 is recycled to the separator overhead stream in line 119. In an embodiment using a PSA unit, the recycle stream in line 207 is a tail gas stream. In another embodiment using a TSA unit, the recycle stream in line 207 is a spent regeneration stream. In an exemplary embodiment, the tail gas stream in line 207 may be passed through a sulfur guard bed 211 to provide a treated tail gas stream in line 212. The sulfur guard bed 211 may treat the tail gas stream in line 207 to remove the hydrogen sulfide that may be present in the tail gas stream. The sulfur guard bed 211 may remove the hydrogen sulfide from both the net gas and tail gas loop. Thes sulfur guard bed 211 may help in preventing corrosion in the tail gas system that may occur in the presence of water vapors, particularly in the event of water ingress from the oxygen stripper 102 and/or the deoxygenated stream in line 103. The treated tail gas stream in line 212 may be recycled to the hydrogen compressor section 123′. In an embodiment, the treated tail gas stream in line 212 may be recycled to the net gas stream in line 122 to provide the combined recycle stream in line 205.

Typically, hydrogen recovery from adsorption unit is about 90%, so it is necessary to recycle 100% of the recycle gas to recover all the hydrogen product while meeting its purity requirement of 1 wtppm BTX limit. The adsorption unit can be the PSA unit or the TSA unit. The PSA unit operates at a pressure of about 507 psig (3500 kPag) to about 1160 psig (8000 kPag), preferably about 507 psig (3500 kPag) to about 725 psig (5000 kPag), at the feed inlet, and in case the separator operates at a pressure of less than about 50 psig (345 kPag), tail gas stream can be recycled without the need of a tail gas compressor. The TSA unit operates at a regeneration temperature of about 200° C. (392° F.) to about 450° C. (842° F.), preferably about 200° C. (392° F.) to about 300° C. (572° F.).

In an embodiment, the adsorption unit 206 is the PSA unit. The PSA unit can include a plurality of adsorption beds containing an adsorbent selective for the separation of hydrogen from the aromatics. Preferably, the PSA unit 206 operates at a feed pressure of about 435 psig (3000 kPag) to about 1160 psig (8000 kPag), preferably about 435 psig (3000 kPag) to about 798 psig (5500 kPag), preferably about 508 psig (3500 kPag) to about 725 psig (5000 kPag). The tail gas pressure is about 4.35 psig (30 kPag) to 72.5 psig (500 kPag), preferably about 4.35 psig (30 kPag) to 43.5 psig (300 kPag). The PSA unit comprises a tail gas compressor to compress the tail gas stream. In an embodiment, the PSA unit can operate without a tail gas compressor. The PSA unit operates without a tail gas compressor when the separator (118) operates at a pressure of less than 50 psig (345 kPa). Often, each adsorption bed within the adsorption zone undergoes, on a cyclic basis, high pressure adsorption, optional co-current depressurization to intermediate pressure levels with the release of product from void spaces, countercurrent depressurization to lower desorption pressure with the release of desorbed gas from the feed end of the adsorption bed, with or without purge of the bed, and repressurization to higher adsorption pressure. This process may also include an addition to this basic cycle sequence, such as a co-current displacement step, or co-purge step in the adsorption zone following the adsorption step in which the less readily adsorbable component, or hydrogen, is essentially completely removed therefrom by displacement with an external displacement gas introduced at the feed end of the adsorption bed. The adsorption zone may then be counter-currently depressurized to a desorption pressure that is at or above atmospheric pressure with the more adsorbable component being discharged from the feed end thereof. The combined gas stream produced during the countercurrent depressurization step and the purge step is typically referred to as the tail gas stream. The tail gas recycle stream comprises some amount of hydrogen, BTX, and hydrogen sulfide.

In another embodiment, the adsorption unit 206 is the TSA unit. The TSA unit can include a plurality of adsorption beds containing an adsorbent selective for the separation of hydrogen from the aromatics. The TSA unit operates at a regeneration temperature of about 200° C. to about 450° C., preferably about 200° C. to 300° C. The TSA process includes two basic steps of adsorption and regeneration. In the adsorption step, contaminants are removed by being adsorbed on the solid adsorbent and then the treated stream leaves the unit with contaminant levels below the required specification limit or further treatment is necessary. In the regeneration step, contaminants are desorbed from the solid molecular sieve material by means of a regeneration stream (typically gas). The regeneration step consists of two major parts-heating and cooling. In the heating part of the process, the regeneration stream, which is contaminant free, is heated to an elevated temperature and flows over the adsorbent. Due to the heat of the gas, mainly resulting from heat of desorption, and the difference in partial pressure of the contaminants on the adsorbent and in the regeneration gas stream, the contaminants desorb from the solid material and leave the unit with the spent regeneration gas. A cooling step is then necessary. As a result of the heating step the adsorbent heats up. To prepare the material again for the next adsorption step and since adsorption is favored at lower temperatures than desorption, the adsorbent needs to be cooled by means of a stream typically flowing over the molecular sieve at a temperature very close to the feed stream temperature. The spent regeneration stream comprises some amount of hydrogen, BTX, and hydrogen sulfide.

The adsorption unit 206 can use any adsorbent material selective for the separation of hydrogen from hydrocarbons in the adsorbent beds. Suitable adsorbents can include one or more crystalline molecular sieves, activated carbons, activated clays, silica gels, activated aluminas, and combinations thereof. Preferably, the adsorbents are one or more of an activated carbon, an alumina, an activated alumina, and a silica gel.

The separator bottom stream in line 120, a first liquid product in line 134′, a second liquid product stream in line 135′, and the discharge drum bottom stream in line 133′ are combined to form a combined liquid stream in line 137′ and sent to the toluene recovery section 500 as described in FIG. 1.

### EXAMPLE

A simulation study was performed to determine the effect of the present disclosure in increasing the purity of hydrogen in a methylcyclohexane dehydrogenation process. The mass flow rates are in lbs/hr. The toluene rate in the product is 252975 lbs/hr (114747 kg/hr). The comparison is shown in the Table.

TABLE

| | | | | | |
|---|---|---|---|---|---|
| Separator liquid to | 0% | 0% | 30% | 30% | 50% |
| Recontact drum | | | | | |
| Chiller outlet | 15° C. | −17° C. | 15° C. | −17° C. | −17° C. |
| temperature, ° C. | | | | | |
| Chiller duty, MMBtu/hr | 1.495 | 1.742 | 2.958 | 4.878 | 6.879 |
| Toluene loss in the | 0.24% | 0.032% | 0.28% | 0.04% | 0.05% |
| hydrogen product | | | | | |
| Mass Flows, kg/hr | | | | | |
| Hydrogen | 7580.79 | 7580.89 | 7571.73 | 7574.08 | 7569.41 |
| Methylcyclohexane | 20.32 | 4.01 | 9.58 | 1.87 | 1.92 |
| Toluene | 610.85 | 81.14 | 708.45 | 106.12 | 116.45 |

As can be seen from the above table, when the chiller outlet temperature is −17° C., the loss of toluene in the hydrogen product is reduced to 0.032% from 0.24% at 15° C.

### SPECIFIC EMBODIMENTS

While the following is described in conjunction with specific embodiments, it will be understood that this description is intended to illustrate and not limit the scope of the preceding description and the appended claims.

A first embodiment of the present disclosure is a process for recovering hydrogen comprising, dehydrogenating a hydrogenated feed stream in a dehydrogenation reactor zone to provide a dehydrogenated stream; separating the dehydrogenated stream in a separator to produce a net gas stream comprising hydrogen and a separator bottom liquid stream comprising dehydrogenated product; compressing the net gas stream in a hydrogen compressor section comprising one or more compressors to produce a compressed stream; cooling the compressed stream in a vapor economizer to produce a cooled compressed stream; chilling the cooled compressed stream in a chiller unit to produce a chilled stream; and separating the chilled stream in a discharge drum to recover a discharge drum overhead stream comprising a hydrogen product stream. An embodiment of the present disclosure is one, any or all of prior embodiments in this paragraph up through the first embodiment in this paragraph, wherein the chilled stream is at a temperature of −27° C. to 17° C. An embodiment of the present disclosure is one, any or all of prior embodiments in this paragraph up through the first embodiment in this paragraph further comprising cooling the compressed stream in a discharge cooler to produce a two-phase compressed stream comprising liquid and vapor phase. An embodiment of the present disclosure is one, any or all of prior embodiments in this paragraph up through the first embodiment in this paragraph further comprising stripping the hydrogenated feed stream in an oxygen stripper to remove one or both of oxygen and oxygenated hydrocarbons. An embodiment of the present disclosure is one, any or all of prior embodiments in this paragraph up through the first embodiment in this paragraph further comprising combining the hydrogenated feed stream with a recycle hydrogen stream to provide a combined feed stream; and pre heating the combined feed stream in a combined heat exchanger by heat exchange with the dehydrogenated effluent stream. An embodiment of the present disclosure is one, any or all of prior embodiments in this paragraph up through the first embodiment in this paragraph further comprising compressing a separator overhead stream in a recycle compressor to produce the recycle hydrogen stream and the net gas stream. An embodiment of the present disclosure is one, any or all of prior embodiments in this paragraph up through the first embodiment in this paragraph further comprising heating the discharge drum overhead stream in vapor economizer before sending it to a chloride treater to produce high purity hydrogen product gas. An embodiment of the present disclosure is one, any or all of prior embodiments in this paragraph up through the first embodiment in this paragraph the dehydrogenated product comprises toluene.

A second embodiment of the present disclosure is a process for recovering hydrogen from a dehydrogenated effluent stream, comprising separating the dehydrogenated effluent stream, in a separator, to produce a separator overhead stream comprising hydrogen and a separator bottom stream comprising dehydrogenated product; compressing the separator overhead stream in a hydrogen compressor section to produce a compressed stream; cooling the compressed stream in a vapor economizer by heat exchange with a discharge drum overhead stream to produce a cooled compressed stream; chilling the cooled compressed stream in a chiller to produce a chilled stream; and separating the chilled stream in a discharge drum to produce the discharge drum overhead stream comprising a hydrogen product stream. An embodiment of the present disclosure is one, any or all of prior embodiments in this paragraph up through the second embodiment in this paragraph, wherein the chilled stream is at a temperature of −27° C. to 17° C. An embodiment of the present disclosure is one, any or all of prior embodiments in this paragraph up through the second embodiment in this paragraph further comprising cooling the compressed stream in a discharge cooler to produce two-phase compressed stream comprising liquid and vapor. An embodiment of the present disclosure is one, any or all of prior embodiments in this paragraph up through the second embodiment in this paragraph further comprising dehydrogenating a hydrogenated feed stream in a dehydrogenation reaction zone to provide the dehydrogenated effluent stream. An embodiment of the present disclosure is one, any or all of prior embodiments in this paragraph up through the second embodiment in this paragraph further comprising stripping the methylcyclohexane stream in an oxygen stripper to produce the hydrogenated feed stream. An embodiment of the present disclosure is one, any or all of prior embodiments in this paragraph up through the second embodiment in this paragraph further comprising heating the discharge drum overhead stream in vapor economizer before sending it to a chloride treater to produce high purity hydrogen product gas.

A third embodiment of the present disclosure is a process for recovering hydrogen, comprising compressing a net gas stream together with a tail gas recycle stream in a hydrogen compressor section to produce a compressed stream; recovering hydrogen from the compressed stream in a pressure swing adsorption (PSA) unit to produce a hydrogen product stream and the tail gas recycle stream; and completely recycling the tail gas recycle stream to the hydrogen compressor section. An embodiment of the present disclosure is one, any or all of prior embodiments in this paragraph up through the third embodiment in this paragraph, wherein the PSA unit operates at a feed pressure of 35 barg (3500 kPa) to 80 barg (8000 kPa). An embodiment of the present disclosure is one, any or all of prior embodiments in this paragraph up through the third embodiment in this paragraph, wherein the PSA unit operates at tail gas pressure of about 0.3 barg (30 kPa) to about 5 barg (500 kPa). An embodiment of the present disclosure is one, any or all of prior embodiments in this paragraph up through the third embodiment in this paragraph, further comprising dehydrogenating a hydrogenated stream in a dehydrogenation reactor zone to provide a dehydrogenated stream; separating the dehydrogenated stream in the separator to produce the net gas stream comprising hydrogen and a separator bottom stream comprising dehydrogenated product. An embodiment of the present disclosure is one, any or all of prior embodiments in this paragraph up through the third embodiment in this paragraph wherein the PSA unit operates without a tail gas compressor, when the separator operates at a pressure of less than 50 psig (345 kPa). An embodiment of the present disclosure is one, any or all of prior embodiments in this paragraph up through the third embodiment in this paragraph further comprising separating the compressed stream in a discharge drum to produce a discharge drum overhead stream and a discharge drum bottom stream. An embodiment of the present disclosure is one, any or all of prior embodiments in this paragraph up through the third embodiment in this paragraph further comprising sending the discharge drum overhead stream to a chloride treater to remove chloride from the discharge drum overhead stream. An embodiment of the present disclosure is one, any or all of prior embodiments in this paragraph up through the third embodiment in this paragraph stripping a hydrogenated feed stream in an oxygen stripper to remove oxygenates. An embodiment of the present disclosure is one, any or all of prior embodiments in this paragraph up through the third embodiment in this paragraph pre-heating the hydrogenated feed stream in a combined heat exchanger by heat exchange with the dehydrogenated effluent stream.

A fourth embodiment of the present disclosure is an apparatus for recovering hydrogen, comprising a hydrogen compressor section in downstream communication with a net gas line and a recycle line; a recycle line in downstream communication with an adsorption unit. An embodiment of the present disclosure is one, any or all of prior embodiments in this paragraph up through the fourth embodiment in this paragraph, wherein the adsorption unit is a temperature swing adsorption (TSA) unit and the recycle line is a spent regeneration line from the TSA unit. An embodiment of the present disclosure is one, any or all of prior embodiments in this paragraph up through the fourth embodiment in this paragraph, wherein the TSA unit operates at a regeneration temperature of about 200° C. to about 400° C., preferably about 200° C. to about 300° C. An embodiment of the present disclosure is one, any or all of prior embodiments in this paragraph up through the fourth embodiment in this paragraph further comprising a dehydrogenation reactor zone in communication with a hydrogenated feed line; and a separator in communication with the dehydrogenation reactor zone. An embodiment of the present disclosure is one, any or all of prior embodiments in this paragraph up through the fourth embodiment in this paragraph further comprising a discharge drum in communication with the hydrogen compressor section; and the TSA unit in downstream communication with the discharge drum. An embodiment of the present disclosure is one, any or all of prior embodiments in this paragraph up through the fourth embodiment in this paragraph further comprising further comprising a chloride treater in downstream communication with the discharge drum. An embodiment of the present disclosure is one, any or all of prior embodiments in this paragraph up through the fourth embodiment in this paragraph further comprising a combined heat exchanger in communication with the dehydrogenation reactor zone.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the present disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

1. A process for recovering hydrogen comprising:
dehydrogenating a hydrogenated feed stream in a dehydrogenation reactor zone to provide a dehydrogenated effluent stream;
separating the dehydrogenated effluent stream in a separator to produce a net gas stream comprising hydrogen and a separator bottom liquid stream comprising dehydrogenated product;
compressing the net gas stream in a hydrogen compressor section comprising one or more compressors to produce a compressed stream;
cooling the compressed stream in a vapor economizer to produce a cooled compressed stream;
chilling the cooled compressed stream in a chiller unit to produce a chilled stream; and
separating the chilled stream in a discharge drum to recover a discharge drum overhead stream comprising a hydrogen product stream.

2. The process of claim 1, wherein the chilled stream is at a temperature of −27° C. to 17° C.

3. The process of claim 1 further comprising cooling the compressed stream in a discharge cooler to produce a two-phase compressed stream comprising liquid and vapor phase.

4. The process of claim 1 further comprising stripping the hydrogenated feed stream in an oxygen stripper to remove one or both of oxygen and/or oxygenated hydrocarbons.

5. The process of claim 1 further comprising combining the hydrogenated feed stream with a recycle hydrogen stream to provide a combined feed stream; and
preheating the combined feed stream in a combined heat exchanger by heat exchange with the dehydrogenated effluent stream.

6. The process of claim 1 further comprising compressing a separator overhead stream in a recycle compressor to produce a recycle hydrogen stream and the net gas stream.

7. The process of claim 1 further comprising heating the discharge drum overhead stream in the vapor economizer before treating in a chloride treater to produce high purity hydrogen product stream.

8. The process of claim 1 wherein the dehydrogenated effluent stream comprises toluene.

9. A process for recovering hydrogen from a dehydrogenated effluent stream comprising:
separating the dehydrogenated effluent stream, in a separator, to produce a separator overhead stream comprising hydrogen and a separator bottom stream comprising dehydrogenated product;
compressing the separator overhead stream in a hydrogen compressor section to produce a compressed stream;
cooling the compressed stream in a vapor economizer by heat exchange with a discharge drum overhead stream to produce a cooled compressed stream;
chilling the cooled compressed stream in a chiller to produce a chilled stream; and
separating the chilled stream in a discharge drum to produce the discharge drum overhead stream comprising a hydrogen product stream.

10. The process of claim 9, wherein the chilled stream is at a temperature of −27° C. to 17° C.

11. The process of claim 9 further comprising dehydrogenating a hydrogenated feed stream in a dehydrogenation reaction zone to provide the dehydrogenated effluent stream.

12. The process of claim 9 further comprising stripping the hydrogenated feed stream in an oxygen stripper to remove oxygenates.

13. The process of claim 9 further comprising heating the discharge drum overhead stream in vapor economizer before treating in a chloride treater to produce high purity hydrogen product stream.

14. A process for recovering hydrogen comprising:
compressing a net gas stream together with a tail gas recycle stream in a hydrogen compressor section to produce a compressed stream;
recovering hydrogen from the compressed stream in a pressure swing adsorption (PSA) unit to produce a hydrogen product stream and the tail gas recycle stream; and
completely recycling the tail gas recycle stream to the hydrogen compressor section.

15. The process of claim 14, wherein the PSA unit operates at a feed pressure of 35 barg (3500 kPa) to 80 barg (8000 kPa) and a tail gas pressure of about 0.3 barg (30 kPa) to about 5 barg (500 kPa).

16. The process of claim 14 further comprising:
dehydrogenating a hydrogenated stream in a dehydrogenation reactor zone to provide a dehydrogenated effluent stream; and
separating the dehydrogenated effluent stream in the separator to produce the net gas stream comprising hydrogen and a separator bottom stream comprising dehydrogenated product.

17. The process of claim 14, wherein the PSA unit operates without a tail gas compressor, when the separator operates at a pressure of less than about 50 psig (345 kPa).

18. The process of claim 14 further comprising separating the compressed stream in a discharge drum to produce a discharge drum overhead stream and a discharge drum bottom stream.

19. The process of claim 14 further comprising stripping a hydrogenated feed stream in an oxygen stripper to remove oxygenates.

20. The process of claim 14 further comprising pre-heating the hydrogenated feed stream in a combined heat exchanger by heat exchange with the dehydrogenated effluent stream.
